(19) United States
(12) Patent Application Publication
(10) Pub. No.: US 2024/0182840 A1
(43) Pub. Date: Jun. 6, 2024

(54) DEVICE FOR CONTINUOUS VIRUS INACTIVATION

(71) Applicant: Sartorius Stedim Biotech GmbH, Goettingen (DE)

(72) Inventors: Jan Schaefer, Edermuende (DE); Stefan Weisshaar, Adelebsen (DE); Alexander Helling, Goettingen (DE); Mario Deuse, Bovenden (DE); Bernhard Diel, Dransfeld (DE); Alexander Tappe, Goettingen (DE)

(21) Appl. No.: 18/283,403

(22) PCT Filed: Mar. 21, 2022
(86) PCT No.: PCT/EP2022/057365

§ 371 Date: Sep. 21, 2023

(30) Foreign Application Priority Data
Mar. 24, 2021 (DE) 10 2021 107 394.4

(51) Int. Cl. C12M 1/12; C12M 1/00; C12M 1/36; C12M 3/00

(52) U.S. Cl. CPC C12M 37/00; C12M 23/40; C12M 23/44; C12M 27/20; C12M 41/48

(57) ABSTRACT

Embodiments include a device for continuous virus inactivation during a protein production process comprising a first and a second fluid inlet, a first mixer, and a fluid outlet. A first liquid stream containing a target protein is introducible into the device through the first fluid inlet and is combinable in a predefined volume ratio with a second, virus-inactivating liquid stream introducible into the device through the second fluid inlet to form a third, reactive liquid stream which is conducted through the first mixer for mixing in order to generate predefined, virus-inactivating conditions. The device further comprises a head part and, downstream of the first mixer and upstream of the fluid outlet, a residence time arrangement fluidically connected to the head part for provision of a minimum residence time of the third stream, wherein the head part and the residence time arrangement are rigidly fastened to each other.

### CROSS-REFERENCE TO RELATED APPLICATIONS

This application is a national stage application under 35 U.S.C. 371 of International Patent Application Serial No. PCT/EP2022/057365, entitled “Device for Continuous Virus Inactivation,” filed Mar. 21, 2022, which claims priority from German Patent Application No. DE 10 2021 107 394.4, filed Mar. 24, 2021, the disclosure of which is incorporated herein by reference.

### FIELD OF THE TECHNOLOGY

Various embodiments relate to a device for continuous virus inactivation, to a residence time arrangement, to a residence time arrangement, to a method for continuous virus inactivation using a proposed device, and to the use of a proposed device or residence time arrangement for implementation of a protein production process.

### BACKGROUND

The device in question for continuous virus inactivation is used as part of the production and/or quality control of biopharmaceutical products, in particular proteins, in a bioprocess, whereby infection of patients by potential viral contamination is to be prevented.

The known device and the known method for continuous virus inactivation (WO 2017/156355 A1), from which some embodiments proceed, serves for the continuous inactivation of viruses during a protein production process, wherein a liquid stream containing a target protein is combined with a virus-inactivating liquid stream in a predefined volume ratio in order to generate a reactive liquid stream. Said reactive liquid stream is then conducted into a static mixer for mixing. After the static mixer, the reactive liquid stream can be conducted into a flexible tube or into some other intermediate element of any type and construction to maintain the resultant pH, before the virus inactivation is ended by the addition of base, there being no performance of feedback control by adjustment of the volume ratio or changing of the virus-inactivating liquid stream.

As a result, the known device for continuous virus inactivation is associated with a high degree of complexity with regard to assembly and handling owing to the large number of individual components. The large number of components required for the method accordingly occupy a relatively large space. In general, performing the known method for continuous virus inactivation proves relatively complicated.

### SUMMARY

The problem addressed by various embodiments is that of designing and developing the known method for continuous virus inactivation in such a way that performance thereof is simplified.

For a device for continuous virus inactivation, the above problem is solved by various features described herein.

“Viruses” are infectious organic structures which spread outside cells by transmission as virions, but can replicate only within a suitable host cell as viruses. These include viruses that can infect bacteria, so-called “bacteriophages” or “phages”, and those that can infect humans and/or animals. The viruses to be inactivated may have been added to the bioprocess either by an external source, in particular by contamination, or by internal sources, in particular produced by the cell line used for the bioprocess.

Here, the term “bioprocess” means biotechnological and biopharmaceutical processes involved in the preparation of therapeutic bioproducts, such as vaccines, biologics or components for cell or gene therapy, or nontherapeutic bioproducts such as pigments, biofuels or nutrients. Such bioproducts can be produced by living cells, or the cell itself can be the bioproduct, or the bioproduct can be the result of cell-free production based on cell components of either natural or nonnatural origin.

What can be essential, for some embodiments, is the fundamental consideration that the device for continuous virus inactivation comprises a head part for generation of a reactive liquid stream and a residence time arrangement for provision of a minimum residence time of the reactive liquid stream within the device. The residence time arrangement and the head part arranged on an end face of the residence time arrangement are rigidly fastened to each other.

The particular structure of the proposed device for continuous virus inactivation has the advantage that the device is modular and therefore particularly compact and, above all, flexible, which is associated per se with, firstly, an increase in the possible applications and, secondly, a reduction in structural complexity and in required space.

Specifically, it is proposed that the device comprises a head part for combination of two liquid streams, one of which is a liquid stream containing the target protein, that the device comprises, downstream of the first mixer and upstream of the fluid outlet, a residence time arrangement fluidically connected to the head part for provision of a minimum residence time of the third, reactive liquid stream within the device, that the head part and the residence time arrangement are rigidly fastened to each other, and that the head part is arranged on an end face of the residence time arrangement.

In various embodiments, the device for continuous virus inactivation comprises three fluid inlets in order to fulfill the intended function. A neutralizing liquid stream can be introduced into the device via the additional fluid inlet. This embodiment offers the possibility of generating a reactive and an at least partially reneutralized, resultant liquid stream within the device, as a result of which the device has particularly small space requirements without performance also being affected.

In various embodiments, the device has a second mixer downstream of the third fluid inlet. This offers the advantage that the virus-inactivating conditions can be neutralized particularly efficiently directly within the device.

In various embodiments, the head part is designed as an inlet head part for generation of the third, reactive liquid stream. Furthermore, the inlet head part is arranged at one end of the residence time arrangement in order to generate the virus-inactivating conditions. The inlet head part makes it possible to establish the reactive liquid stream directly within the device.

In various embodiments, the head part is designed as an outlet head part for generation of the fifth, resultant liquid stream. Furthermore, the outlet head part is arranged at the other end of the residence time arrangement in order to neutralize the virus-inactivating conditions. The outlet head part therefore makes it possible to neutralize the virus-inactivating conditions directly within the device.

In various embodiments, the first and/or the second fluid inlet and/or the first mixer is integrated into the inlet head part, and/or the third fluid inlet and/or the second mixer is integrated into the outlet head part. This allows a particularly compact design and offers the advantage of reducing structural complexity and reducing the necessary space requirements.

In various embodiments, the first two liquid streams are combined upstream of the first mixer or in the first mixer, and/or the third and the fourth liquid stream are combined upstream of the second mixer or in the second mixer. This embodiment allows the generation of up to five liquid streams within the device, while ensuring optimal mixing.

In various embodiments, the residence time arrangement is in the form of a substantially cylindrical or cuboid body. This offers the advantage that these geometric shapes easily arrangeable and their compact shape improves their handling. A possible embodiment as a one-piece body offers the advantage that the structural complexity of the residence time arrangement is further reduced.

Various embodiments show that the proposed solution allows a high degree of constructional flexibility. This is achieved here by the residence time arrangement comprising an internal channel system as a component necessary for the intended function. This can offer the advantage that the embodiment of the proposed device can be adapted to individual process requirements with regard to complexity.

Various embodiments relate to one or more residence time levels of the internal channel system, which are arranged at least largely transversely to the longitudinal axis of the residence time arrangement. This provides particular structural flexibility, since the number of residence time levels and/or residence time level channels can be adapted to individual process requirements. Furthermore, this variant offers the advantage that venting of the device is relatively simple to realize.

Various embodiments include the respective residence time level as a preassembled or one-piece component. A possible combinability of a plurality of these components offers the advantage of individual adjustment of the residence time within the proposed device and thereby allows a modular setup for optimal scalability.

In various embodiments, the residence time arrangement is sealingly connected to the head part. This offers the advantage of simplified handling of the device as such and further reduces structural complexity.

Various embodiments relate to specifications regarding a sensor within the proposed device. Therefore, it comprises at least one sensor for measurement of a parameter. Furthermore, at least one sensor can be integrated into the head part. This allows particular flexibility regarding use and allows a compact construction.

In various embodiments, the device comprises a separate inlet intermediate plate and/or a separate outlet intermediate plate. They serve for the respective specific transfer of the third, reactive liquid stream and offer the advantage of an optimal fluidic connection.

In various embodiments, the components of the device at least necessary for the intended function form a functional group, wherein the device can comprise only a single functional group or a plurality of functional groups. This optimizes handling during assembly, allows an extension of the residence time within the device and/or increases the inactivatable volume flow.

In various embodiments, the residence time arrangement, the head part, the inlet intermediate plate and/or the outlet intermediate plate are produced in a plastics injection-molding process or in a 3D printing process. This offers particularly cost-effective ways of production.

It may also be noted that the proposed device, as a whole or at least in parts, in particular the residence time arrangement, the inlet head part, the outlet head part, the inlet intermediate plate and/or the outlet intermediate plate, can be readily designed as a disposal component. An appropriate replacement of the device as a whole or of at least the respective part after single use has the advantage that sterility is ensured and any cleaning steps after the end of the process are dispensed with.

Various embodiments provide a residence time arrangement for provision of a predefined minimum residence time of a reactive liquid stream during continuous virus inactivation in a device for continuous virus inactivation, in particular in a proposed device, in particular during an antibody production process, is provided, wherein, when the residence time arrangement is assembled as intended, a liquid stream containing a target protein and having predefined, virus-inactivating conditions is introducible into the residence time arrangement. What is essential is that the residence time arrangement comprises an internal channel system for provision of a minimum residence time of the reactive liquid stream within the residence time arrangement, that the internal channel system comprises at least one channel through which the reactive liquid stream flows during the continuous virus inactivation, and that the liquid stream is deflected at least once within the respective channel. The particular design of the residence time arrangement allows a particularly simple way of establishing a desired minimum residence time. Reference may be made in this respect to all discussions relating to the proposed device for continuous virus inactivation.

In various embodiments, the liquid stream is deflected within the respective channel in such a way that its subsequent flow direction runs transversely to the prior flow direction. This allows a particularly simple way of producing homogeneous mixing.

Various embodiments of the residence time arrangement that have already been described above in relation to the device for continuous virus inactivation are also possible.

Various embodiments provide a residence time arrangement for provision of a predefined minimum residence time of a reactive liquid stream during continuous virus inactivation in a device for continuous virus inactivation, in particular in a proposed device, in particular during an antibody production process, is provided, wherein, when the residence time arrangement is assembled as intended, a liquid stream containing a target protein and having predefined, virus-inactivating conditions is introducible into the residence time arrangement. What is essential here is that the residence time arrangement comprises an internal channel system for provision of a minimum residence time of the third, reactive liquid stream within the residence time arrangement, that the internal channel system comprises at least one channel through which the reactive liquid stream flows during the continuous virus inactivation, that the at least one channel is helically designed for provision of a predefined minimum residence time, and that the residence time arrangement is helically arranged in the intended state. The particular design of the residence time arrangement makes it possible to greatly save installation space and to reduce the required production area. Reference may be made in this respect to all discussions relating to the proposed device for continuous virus inactivation.

Some embodiments relate to the residence time arrangement being made of a rigid or flexible material and thus allows particular flexibility in the realization of the method.

Various embodiments relate to the presence of at least one pig for cleaning and/or inspection of the residence time arrangement. This makes it particularly simple to service and maintain the residence time arrangement. On the other hand, the use of pigs makes it possible to divide the liquid stream, and so it is possible to produce different volume components which can have different properties.

In various embodiments, the at least one residence time arrangement is assigned a corresponding holding arrangement. This opens up the possibility of the residence time arrangement being arranged helically on the holding arrangement when assembled as intended for even greater saving of installation space. The residence time arrangement and the holding arrangement assigned thereto jointly form a residence time system.

Some embodiments relate the holding arrangement with a rectangular or rounded base surface. This can offer the advantage of a particularly simple design of the holding arrangement which, at the same time, increases user-friendliness.

Various embodiments provide the residence time arrangement that have already been described above in relation to the device for continuous virus inactivation.

Various embodiments provide a method for continuous virus inactivation during a protein production process, in particular an antibody production process, using a proposed device and optionally a proposed residence time arrangement is provided, wherein a first liquid stream containing a target protein is introduced into the device through the first fluid inlet and is combined in a precisely predefined volume ratio with a second, virus-inactivating liquid stream introduced into the device through the second fluid inlet to form a third, reactive liquid stream which is conducted through the first mixer for mixing in order to generate predefined, virus-inactivating conditions, wherein the third, reactive liquid stream is generated in the inlet head part and wherein a minimum residence time of the third, reactive liquid stream within the device is provided by means of the residence time arrangement. Reference may be made in this respect to all discussions relating to the proposed device for continuous virus inactivation and relating to the respective proposed residence time arrangement.

A method using a proposed device simplifies the performance of virus-inactivating processes.

Equipping the proposed device with a residence time arrangement, in particular a proposed residence time arrangement, comprising an internal channel system ensures a greatest possible residence time with only a small space requirement, thereby optimizing the size of the inactivatable volume flow and more efficiently configuring the virus inactivation method. It is crucial that the distribution of the residence times of the individual volume components to be inactivated is as uniform as possible in order to obtain a reproducible inactivation result.

In various embodiments, the virus-inactivating conditions of the method are neutralized by means of a fourth, neutralizing liquid stream introduced into the device. As a result, it is possible for the virus-inactivated product to be directly further processed following the method.

In various embodiments, the method is performed in combination with chromatography methods and/or in combination with filtration methods. As a result, the proposed method offers flexibility in the application thereof and offers direct optimization of protein production processes and thus ultimately of protein products themselves.

Various embodiments provide the use of a proposed device for implementation of a protein production process, in particular an antibody production process. Reference may be made in this respect to all discussions relating to the proposed device for continuous virus inactivation and relating to the respective proposed residence time arrangement.

Various embodiments provide a device for continuous virus inactivation during a protein production process, in particular an antibody production process, comprising a first and a second fluid inlet, each configured to introduce a liquid stream into the device, comprising a first mixer, configured to mix a liquid stream, and comprising a fluid outlet, configured to discharge a liquid stream from the device, wherein, when the device is assembled as intended, a first liquid stream containing a target protein is introducible into the device through the first fluid inlet and is combinable in a precisely predefined volume ratio with a second, virus-inactivating liquid stream introducible into the device through the second fluid inlet to form a third, reactive liquid stream which is conducted through the first mixer for mixing in order to generate predefined, virus-inactivating conditions,
wherein the device comprises a head part for combination of two liquid streams, one of which is the liquid stream containing the target protein, in that the device comprises, downstream of the first mixer and upstream of the fluid outlet, a residence time arrangement fluidically connected to the head part for provision of a minimum residence time of the third, reactive liquid stream within the device, in that the head part and the residence time arrangement are rigidly fastened to each other, and in that the head part is arranged on an end face of the residence time arrangement.

In various embodiments, the device comprises, downstream of the first mixer and in particular the residence time arrangement, a third fluid inlet, configured to introduce a liquid stream into the device, and in that, when the device is assembled as intended, the third, reactive liquid stream is combinable with a fourth, neutralizing liquid stream introducible through the third fluid inlet to form a fifth, resultant liquid stream in order to neutralize the virus-inactivating conditions, further in that the fifth, resultant liquid stream can be discharged from the device through the fluid outlet.

In various embodiments, the device comprises, downstream of the third fluid inlet, a second mixer, configured to mix the fifth, resultant liquid stream.

In various embodiments, the head part is an inlet head part for generation of the third, reactive liquid stream and in that the inlet head part is arranged at one end of the residence time arrangement in order to generate the virus-inactivating conditions.

In various embodiments, the head part is an outlet head part for generation of the fifth, resultant liquid stream and in that the outlet head part is arranged at the other end of the residence time arrangement in order to neutralize the virus-inactivating conditions.

In various embodiments, the first and/or the second fluid inlet and/or the first mixer is integrated into the inlet head part, and/or in that the third fluid inlet and/or the second mixer is integrated into the outlet head part.

In various embodiments, the first liquid stream containing a target protein and the second, virus-inactivating liquid stream are combined upstream of the first mixer or in the first mixer, and/or in that the third, reactive liquid stream and the fourth, neutralizing liquid stream are combined upstream of the second mixer or in the second mixer.

In various embodiments, the residence time arrangement is in the form of a substantially cylindrical or cuboid body and/or in the form of a one-piece body.

In various embodiments, the residence time arrangement comprises an internal channel system which is arranged at least largely parallel to the longitudinal axis of the residence time arrangement and/or an internal channel system which is arranged at least largely transversely, in particular orthogonally, to the longitudinal axis of the residence time arrangement and/or an internal channel system which is at least largely arranged as a worm shaft and/or an internal channel system which is a least largely arranged as a cascade.

In various embodiments, the internal channel system which is arranged at least largely transversely, in particular orthogonally, to the longitudinal axis of the residence time arrangement comprises one or more residence time levels, each of which comprises at least one, in particular exactly one, residence time level channel arranged transversely, in particular orthogonally, to the longitudinal axis of the residence time arrangement.

In various embodiments, the respective residence time level is in the form of a preassembled or one-piece component, such as in that a plurality of said components are combinable with one other, in particular stackable on top of one another, further in some embodiments stackable on top of one another with an angular offset between adjacent components, in order to form the channel system.

In various embodiments, the residence time arrangement is sealingly connected via an interlocking, frictional and/or bonded connection to the head part, in particular the inlet head part and/or the outlet head part, such as in that the head part is in the form of a cap and is separably or inseparably fitted onto the residence time arrangement.

In various embodiments, the device comprises at least one sensor for measurement of a parameter of the third, reactive liquid stream and/or at least one sensor for measurement of a parameter of the fifth, resultant liquid stream, such as in that at least one sensor measures at least one parameter from the group comprising pH, conductivity, temperature, light absorption, light intensity, light scattering or other spectrophotometric properties.

In various embodiments, at least one sensor is integrated into the head part, such as in that the at least one sensor is integrated into the inlet head part and in particular the at least one sensor is arranged downstream of the first mixer and/or in that the at least one sensor is integrated into the outlet head part and in particular the at least one sensor is arranged downstream of the second mixer.

In various embodiments, the device comprises a separate inlet intermediate plate between the inlet head part and the residence time arrangement and/or a separate outlet intermediate plate between the residence time arrangement and the outlet head part for the respective specific transfer of the third, reactive liquid stream, such as in that the inlet intermediate plate separates the interior of the inlet head part from the interior of the residence time arrangement and/or the outlet intermediate plate separates the interior of the residence time arrangement from the interior of the outlet head part, further in some embodiments in that the inlet intermediate plate and/or the outlet intermediate plate has a through opening which, when the device is assembled as intended, fluidically connects the interior of the respective head part to the interior of the residence time arrangement, further in some embodiments in that the through opening produces the only fluidic connection between the interior of the respective head part and the interior of the residence time arrangement.

In various embodiments, the components of the device at least necessary for the intended function, in particular at least comprising the inlet head part, the residence time arrangement and the outlet head part, form a functional group, such as in that the functional group can be in the form of a preassembled or one-piece unit.

In various embodiments, the device comprises only a single functional group or a plurality of parallelly and/or serially arranged functional groups fluidically connected to one another, such as in that, from the plurality of functional groups, individual functional groups or a group of functional groups, in particular each functional group, can be connectable or disconnectable by means of a control arrangement of the device.

In various embodiments, the residence time arrangement, the head part, in particular the inlet head part and/or the outlet head part, the inlet intermediate plate and/or the outlet intermediate plate are produced, in particular individually or together, in a plastics injection-molding process or in a 3D printing process.

Various embodiments provide a residence time arrangement for provision of a predefined minimum residence time of a reactive liquid stream during continuous virus inactivation in a device for continuous virus inactivation, in particular in a device as provided herein, in particular during an antibody production process, wherein, when the residence time arrangement is assembled as intended, a liquid stream containing a target protein and having predefined, virus-inactivating conditions is introducible into the residence time arrangement, wherein the residence time arrangement comprises an internal channel system for provision of a minimum residence time of the reactive liquid stream within the residence time arrangement, in that the internal channel system comprises at least one channel through which the reactive liquid stream flows during the continuous virus inactivation, and in that the liquid stream is deflected at least once within the respective channel.

In various embodiments, the liquid stream is deflected within the respective channel in such a way that its subsequent flow direction runs transversely to the previous flow direction, such as in that the liquid stream is deflected within the respective channel by at least 45°, by at least 90°, by at least 135°, or by 180°.

Various embodiments provide a residence time arrangement as described herein.

Various embodiments provide a residence time arrangement for provision of a predefined minimum residence time of a reactive liquid stream during continuous virus inactivation in a device for continuous virus inactivation, in particular in a device as provided herein, in particular during an antibody production process, wherein, when the residence time arrangement is assembled as intended, a liquid stream containing a target protein and having predefined, virus-inactivating conditions is introducible into the residence time arrangement, wherein the residence time arrangement comprises an internal channel system for provision of a minimum residence time of the third, reactive liquid stream within the residence time arrangement, in that the internal channel system comprises at least one channel through which the reactive liquid stream flows during the continuous virus inactivation, in that the at least one channel is helically designed for provision of a predefined minimum residence time, and in that the residence time arrangement is helically arranged in the intended state.

In various embodiments, the residence time arrangement is made of a rigid or flexible material, such as in that the residence time arrangement is in the form of a tube or flexible tube.

In various embodiments, the residence time arrangement has at least one pig in the internal channel system, such as in the at least one channel of the internal channel system, configured to clean and/or inspect the residence time arrangement.

In various embodiments, the at least one residence time arrangement is assigned a corresponding holding arrangement, in that the residence time arrangement is arranged helically on the holding arrangement when assembled as intended, so that the residence time arrangement and the holding arrangement assigned thereto jointly form a residence time system.

In various embodiments, the holding arrangement has a rectangular, such as square, or rounded, such as round or oval, base surface.

Various embodiments provide a residence time arrangement as described herein.

Various embodiments provide a method for continuous virus inactivation during a protein production process, in particular an antibody production process, using a device as described herein and optionally a residence time arrangement as described herein, wherein a first liquid stream containing a target protein is introduced into the device through the first fluid inlet and is combined in a precisely predefined volume ratio with a second, virus-inactivating liquid stream introduced into the device through the second fluid inlet to form a third, reactive liquid stream which is conducted through the first mixer for mixing in order to generate predefined, virus-inactivating conditions, wherein the third, reactive liquid stream is generated in the inlet head part and wherein a minimum residence time of the third, reactive liquid stream within the device is provided by means of the residence time arrangement.

In various embodiments, the virus-inactivating conditions in the third, reactive liquid stream are neutralized in the outlet head part by means of a fourth, neutralizing liquid stream introduced into the device.

In various embodiments, it is performed in combination with chromatography methods, such as with continuous chromatography methods, and/or in combination with filtration methods, such as tangential flow filtration methods.

Various embodiments provide a use of a device as described herein or of a residence time arrangement as described herein for implementation of a protein production process, in particular an antibody production process.

### BRIEF DESCRIPTION OF THE DRAWINGS

In the following, various aspects will be more particularly elucidated with reference to a drawing depicting merely exemplary embodiments. In the drawing

FIG. 1 shows two exemplary embodiments of a proposed device for continuous virus inactivation in a sectional view with a) a static mixer or b) a dynamic mixer,

FIG. 2 shows a further exemplary embodiment of a proposed device in a) a perspective view and b) an exploded view,

FIG. 3 shows an exemplary embodiment of a residence time arrangement of a proposed device according to FIG. 2 in a perspective view,

FIG. 4 shows a further exemplary embodiment of a proposed device in a) a perspective view and b) an exploded view,

FIG. 5 shows an exemplary embodiment of a residence time level of a proposed device according to FIG. 4 in a) a plan view and b) a bottom view of a residence time level,

FIG. 6 shows a further exemplary embodiment of a proposed device in a) a perspective view and b) an exploded view,

FIG. 7 shows a further exemplary embodiment of a proposed device in a) a perspective view and b) an exploded view,

FIG. 8 shows the proposed device according to FIG. 1 having multiple parallelly and/or serially connected proposed devices in a schematic overview,

FIG. 9 shows an exemplary embodiment of a proposed residence time arrangement a) designed as a bag and b) designed as a cassette,

FIG. 10 shows an exemplary embodiment of a proposed residence time arrangement a) in a design with two chambers and b) designed as a “revolver system”,

FIG. 11 shows an exemplary embodiment of a proposed residence time arrangement a) designed as a two-part deep-drawn part, b) in a serially connected design with multiple cassettes, and c) in a stacked design with multiple cassettes,

FIG. 12 shows an exemplary embodiment of a proposed residence time arrangement a) in a design with a helically arranged channel and b) in a helical arrangement of the residence time arrangement as such, and

FIG. 13 shows an exemplary embodiment of a proposed residence time arrangement a) in a design with at least one pig, b) designed as a residence time system with a holding arrangement having a rectangular base surface, and c) in a design of the holding arrangement with a round base surface.

### DETAILED DESCRIPTION

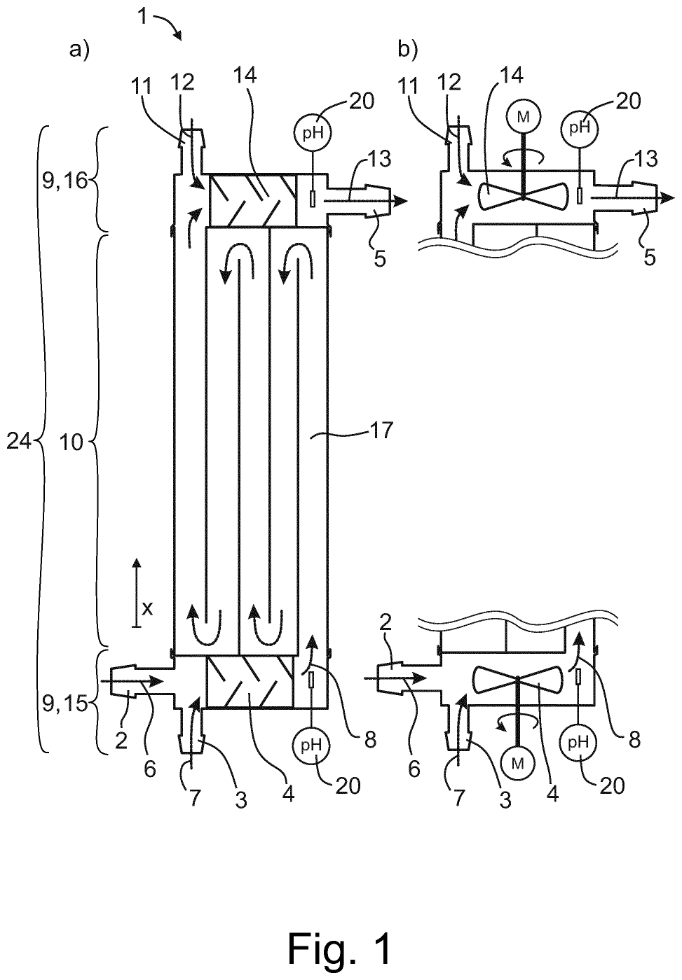

FIG. 1 depicts a proposed device 1 for continuous virus inactivation. It is used in the production and/or quality control of biopharmaceutical products, such as during the production of a protein by means of a bioprocess. Such proteins may be, for example, growth factors, hormones, enzymes and in particular antibodies, antibody derivatives or the like. The proposed device 1 can be used to ensure that a biopharmaceutical product does not contain any active virus particles of any type above a particular threshold defined by, for example, the manufacturer and/or regulatory authorities, etc., in particular does not contain any active virus particles at all.

The target protein can originate from a bioreactor either directly or indirectly, in particular after processing steps, in particular downstream process steps, such as filtration, precipitation and/or chromatographic separation steps and the like, have been performed. Such chromatographic steps can be, for example, affinity chromatography steps, in particular affinity chromatography steps using protein A.

The proposed device 1 comprises a first and a second fluid inlet 2, 3, each configured to introduce a liquid stream into the device 1.

Furthermore, it comprises a first mixer 4, configured to mix a liquid stream, and a fluid outlet 5, configured to discharge a liquid stream from the device 1. Here, a mixer refers to a component which comprises one or more rigid or movable guide structures protruding into the flow cross-section. A mixer must therefore be distinguished from lines themselves and connection points or similar structures at which at least two lines are combined. The mixer 4 can be static (FIG. 1a) or dynamic (FIG. 1b), in particular in the form of a radial or laminar static mixer or stirrer or the like.

When the device 1 is assembled as intended, a first liquid stream 6 containing a target protein is introducible into the device 1 through the first fluid inlet 2. This first liquid stream 6 contains as target protein the biopharmaceutical product, such as an antibody, and process-related viruses. It is then combined in a precisely predefined volume ratio with a second, virus-inactivating liquid stream 7 introducible into the device through the second fluid inlet 3 to form a third, reactive liquid stream 8.

Here, “predefined volume ratio” means that the volume ratio of the liquid streams to be mixed can be already defined before introduction into the device 1 or can be adjusted during the intended use of the device 1. In some embodiments, the volume ratio of the liquid streams to be mixed is defined and/or adjusted by the user. A possible adjustment can be made on the basis of the measured parameters of at least one sensor 20, which will be described in the following, thereby allowing reactive control of the volume ratio.

Here, “reactive liquid stream” means the liquid stream which is formed by the combination of the first liquid stream 6 containing a target protein with the second, virus-inactivating liquid stream 7 and in which the virus-inactivating reaction takes place.

The second, virus-inactivating liquid stream 7 has virus-inactivating conditions, in particular a pH of less than 3, as the necessary property for fulfilling the intended function. The virus-inactivating conditions, in particular the pH, of the virus-inactivating liquid stream 7 is/are chosen such that the third, reactive liquid stream 8 which results following combination with the first liquid stream 6 containing a target protein also has virus-inactivating conditions, in particular a pH of 3 to 3.8 and/or a detergent concentration between 0.05% and 10% (v/v). Such conditions lead to effective virus inactivation without damaging the particular product of the bioprocess, in particular proteins. The pH is achieved by addition of an acid such as lactic acid, ascorbic acid, acetic acid, hydrochloric acid, phosphoric acid, citric acid, glycine, succinic acid and/or sulfuric acid or the like. In some embodiments, the virus-inactivating reagent can contain an acid having a titratable group with a pKa between 2.0 and 4.3. The virus-inactivating conditions can be chosen such that the concentration of the acid can be up to 100 mM but still has sufficient buffering properties in order, firstly, to allow effective virus inactivation and, secondly, to avoid damage to the protein product, for example by acid denaturation of the protein. Additionally or alternatively, the virus-inactivating conditions can be generated by a nonionic detergent having a chromophoric group with an absorption peak between 230 nm and 600 nm. Such detergents can be, for example, Triton-X 100 and other polyethylene oxides. The absorption peak of such detergents make it possible to continuously track the detergent concentration through, for example, absorption of ultraviolet light by the chromophoric group, which is a concentration-dependent property.

As depicted in FIG. 1a), the second liquid stream 7 for virus inactivation is mixed with the first liquid stream 6 containing a target protein in a precisely predefined volume ratio of these two liquid streams 6, 7 to one other, so that it can be ensured that that the virus-inactivating conditions are actually present and homogeneously distributed in the third, reactive liquid stream 8 in order to fulfill the intended function. Such a volume ratio can be, in particular, 9:1, with nine parts of a first liquid stream 6 containing a target protein and one part of a second, virus-inactivating liquid stream 7. Additionally or alternatively, the optimal volume ratio is determined individually for the process before it is performed. The third, reactive liquid stream 8 is then conducted through the first mixer 4 for mixing in order to generate the predefined, virus-inactivating conditions.

What is essential in the case of the proposed device 1 is then, first of all, that it comprises a head part 9 for combination of two liquid streams, one of which is the liquid stream 6 containing the target protein. Downstream of the first mixer 4 and upstream of the fluid outlet 5, the device 1 comprises a residence time arrangement 10, fluidically connected to the head part 9, for provision of a minimum residence time of the third, reactive liquid stream 8 within the device 1.

Here, the term “fluidically connected” means a tight connection which enables a fluid to internally reach one region from another region, at least unidirectionally, such as bidirectionally. Here, the fluidic connection is also separated by mechanical release.

Here, “residence time arrangement” means an arrangement which, when assembled as intended, serves to allow a particular volume flow consisting of one or more combined fluid streams to “reside” within said arrangement, by the residence time arrangement being of such a design that the distance to be covered by the volume flow is artificially extended such that it is necessary to cover a distance many times the extent of the component. As already explained above, it is crucial that the distribution of the residence times of the individual volume components to be inactivated must be as uniform as possible in order to obtain a reproducible inactivation result.

Furthermore, it is essential that the head part 9 is arranged on an end face of the residence time arrangement 10 and that both components are rigidly fastened to each other.

Here, the term “end face” is based on the longitudinal axis X of the residence time arrangement 10. The head part 9 can be coaxially or radially offset in relation to the longitudinal axis X of the residence time arrangement 10. Here, “longitudinal axis” means the axis of a geometric body that corresponds to the direction of its greatest extent.

Here, the term “rigidly” means a nondestructively or destructively separable connection between two components that are immovable relative to one another owing to said connection. Here, the expression “fastened to each other” means an engagement with one another and hold relative to one other produced as a result, in particular a mechanical connection without a flexible tube and/or without a tube. The flow direction can occur along both possible directions of the longitudinal axis X of the residence time arrangement 10. Here, preference can be given to the flow direction against the gravitational direction, since it allows more effective venting of the device 1, thereby preventing air bubbles within the system and also consequently preventing impairments in the downstream process.

Here, as shown in FIGS. 1, 2, 4, 6 and 7, the device 1 comprises a third fluid inlet 11 which is configured to introduce a liquid stream into the device 1. It is downstream of the first mixer 4 and in particular the residence time arrangement 10. As a result, when the device 1 is assembled as intended, the third, reactive liquid stream 8 can be combined with a fourth, neutralizing liquid stream 12 introducible through the third fluid inlet 11.

Here, “neutralizing” means the partial or complete termination and/or removal of the virus-inactivating conditions, in particular the reaction of equal amounts of acids, for example 1.5 to 3 M acetic acid or glycine, and bases, for example 1 to 2 M HEPES, pH 8 or Tris, pH 11. Said fourth, neutralizing liquid stream 12 serves for the neutralization, depletion and/or removal of the virus-inactivating conditions. The combination of the third and the fourth liquid stream 8, 12 results in a fifth, resultant liquid stream 13, which can be discharged from the device 1 through the fluid outlet 5 and has a pH, such as a pH between 5 and 8.5, which allows further processing. Here, these two liquid streams 8, 12 are likewise mixed in a precisely predefined volume ratio, so that it can be ensured that the neutralizing conditions are actually present and homogeneously distributed in the fifth, resultant liquid stream 13 in order to fulfill the intended function.

Here, the device 1 comprises, downstream of the third fluid inlet 11, a second mixer 14, configured to mix the fifth, resultant liquid stream 13. This mixer, too, can be in the form of a static or dynamic mixer, in particular a radial or laminar static mixer or stirrer or the like, as can be seen in FIG. 1a) and FIG. 1b).

Here, a head part 9 is designed as an inlet head part 15 for generation of the third, reactive liquid stream 8. Said inlet head part 15 is arranged at one end of the residence time arrangement 10 in order to generate the virus-inactivating conditions. Here, the term “at one end” means the upstream end of the residence time arrangement 10 based on the longitudinal axis X of the residence time arrangement 10.

Irrespective of this, here, a head part 9 can be designed as an outlet head part 16 for generation of the fifth, resultant liquid stream 13. Said outlet head part 16 is arranged at the other end of the residence time arrangement 10 in order to neutralize the virus-inactivating conditions. Here, the term “at the other end” means the downstream end of the residence time arrangement 10 based on the longitudinal axis X of the residence time arrangement 10 and forms the counterpart to the term “at one end”.

As illustrated in FIG. 1, there can be integrated into the inlet head part 15 the first and/or the second fluid inlet 2, 3 and/or the first mixer 4. Additionally or alternatively, there can be integrated into the outlet head part 16 the third fluid inlet 8 and/or the second mixer 14. The two mixers 4, 14 can be, in each case, either static or dynamic. In addition, they can be either the same or different.

Here, the first liquid stream 6 containing target protein and the second, virus-inactivating liquid stream 7 can be combined upstream of the first mixer 4 or in the first mixer 4.

Additionally or alternatively, the third, reactive liquid stream 8 and the fourth, neutralizing liquid stream 12 can be combined upstream of the second mixer 14 or in the second mixer 14.

In addition, the residence time arrangement 10 can be in the form of a substantially cylindrical or cuboid body. Here, “substantially” means that the residence time arrangement 10 can be at least partially, in some embodiments at least largely, cylindrical and/or cuboid and/or that the residence time arrangement 10 can deviate in places from a cylindrical and/or cuboid contour through, for example, projections or indentations, but otherwise follows a cylindrical and/or cuboid contour. In various embodiments, the residence time arrangement 10 is, additionally or alternatively, in the form of a one-piece body. Here, the term “one-piece” means “made in one piece”.

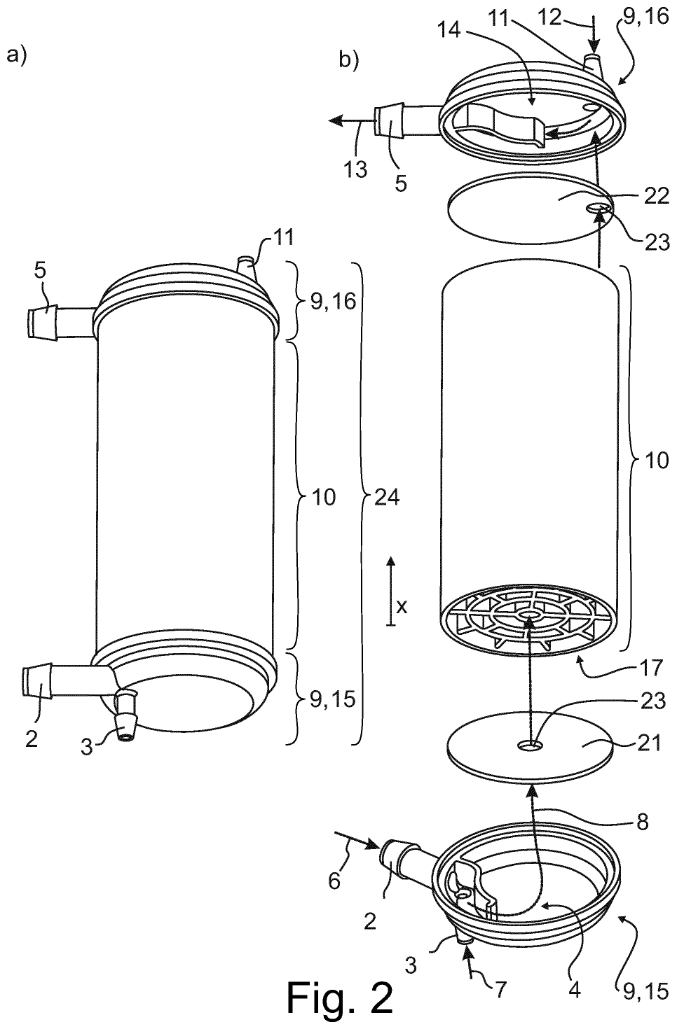

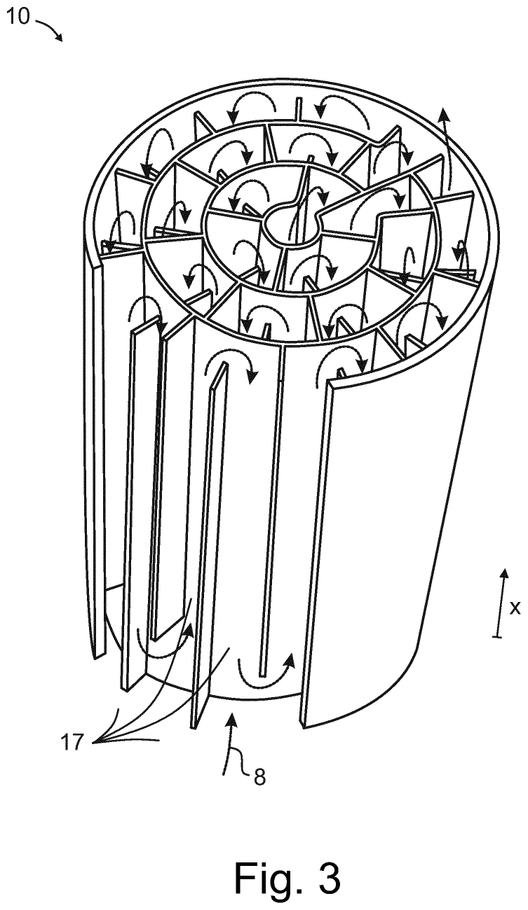

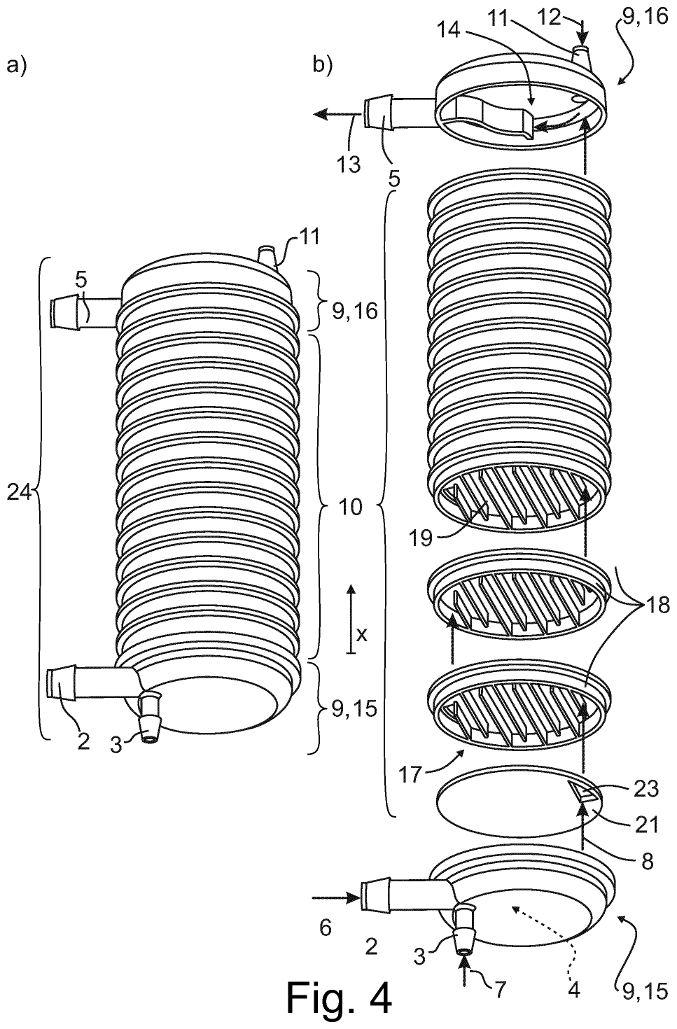

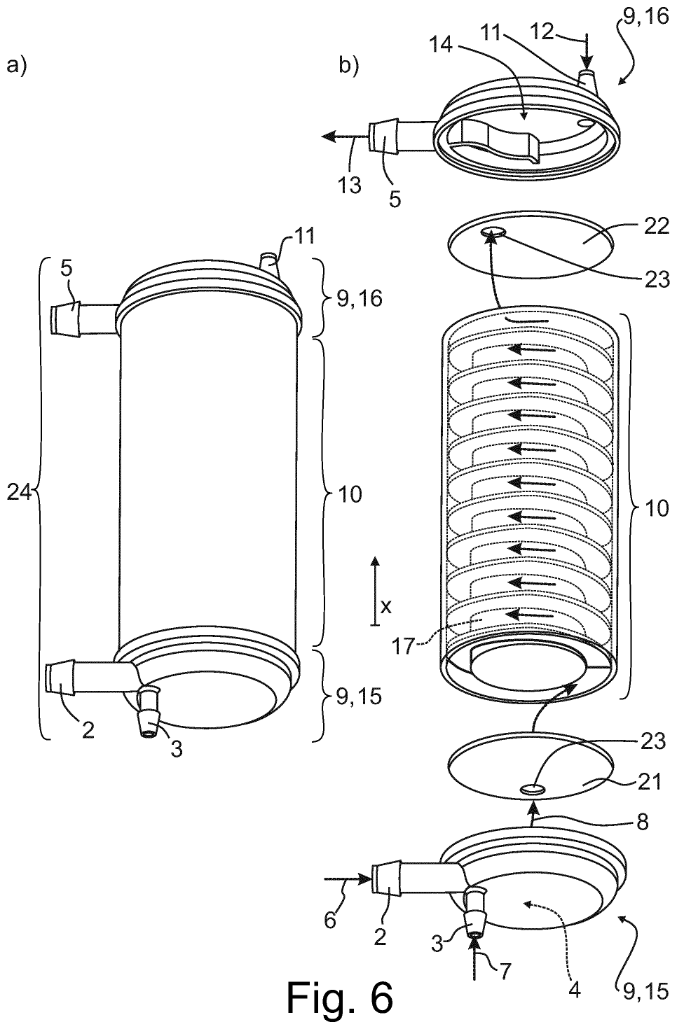

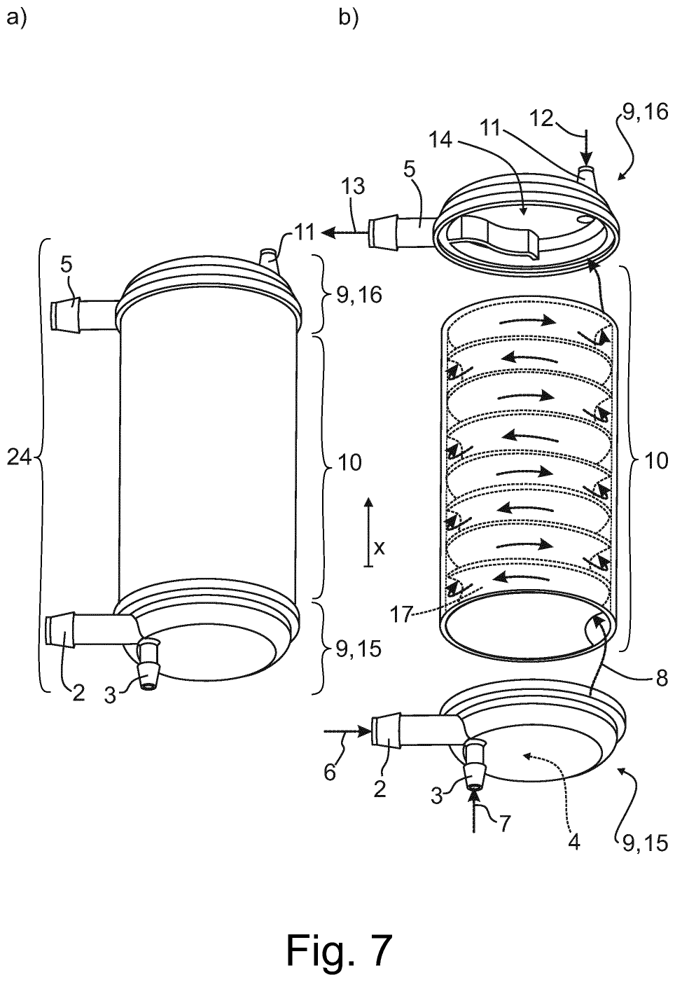

Various embodiments depicted are such that the residence time arrangement 10 comprises an internal channel system 17 in order to fulfill the intended function. Here, the term “internal channel system” means one or more channels, each of which leads from the inlet head part 15 in the direction of the fluid outlet, in particular toward the outlet head part 16. Here, such an internal channel system 17 forms the interior of the residence time arrangement 10. Furthermore, the third, reactive liquid stream 8 can be deflected one or more times, by at least 45°, by at least 90°, by at least 135°, or by 180°, within the respective channel. As a result, one channel can therefore have multiple parallel subsections, in particular at a residence time level 18. In some embodiments, the channel system 17 of the residence time arrangement 10 has an identical cross-sectional area over the predominant part of the channel, such as of all parallel subsections of the respective residence time level channel 18. Said channel system 17 can be arranged at least largely parallel to the longitudinal axis X of the residence time arrangement 10 (see FIG. 2b) and FIG. 3) and/or at least largely transversely, in particular orthogonally, to the longitudinal axis X of the residence time arrangement 10, as shown in FIG. 4b). As per FIG. 6 and FIG. 7, the residence time arrangement 10 can, additionally or alternatively, comprise an internal channel system 17 which is at least largely arranged as a worm shaft and/or at least largely arranged as a cascade in order to fulfill the intended function.

Here, the term “worm shaft” means the shape of a shaft with a helical thread along the longitudinal axis X of the residence time arrangement 10 for forwarding of fluid movements. Here, the term “cascade” means a shape which falls or rises over multiple stages and which extends along the longitudinal axis X of the residence time arrangement 10. As depicted in FIG. 7b), the channels of said channel system 17 are planar, with their diameter or width, in some embodiments, corresponding to the diameter or width of the residence time arrangement 10. This design is particularly advantageous because it promotes the venting of the residence time arrangement 10. Here, the residence time arrangement 10 can be cylindrical. However, a cuboid shape would also be conceivable. This applies to all the examples shown here.

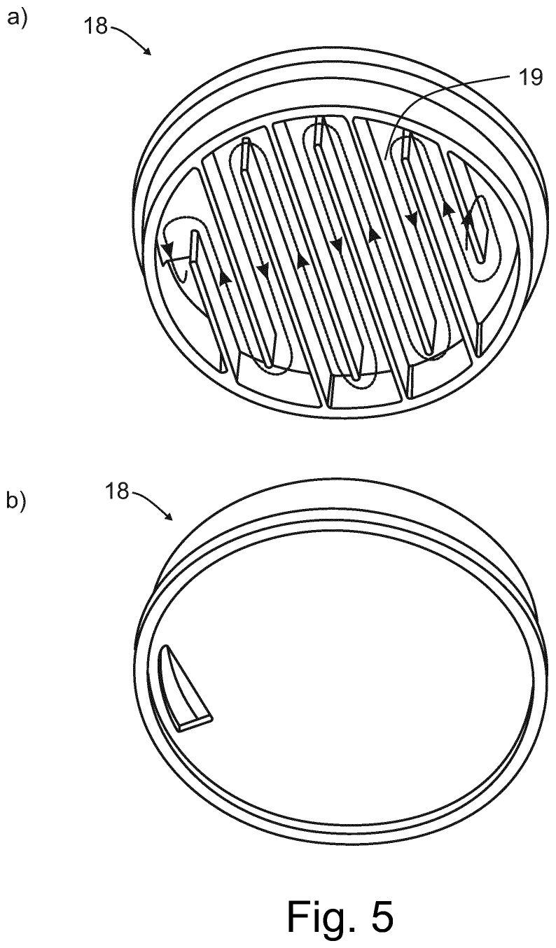

The internal channel system 17 that is arranged at least largely transversely, in particular orthogonally, to the longitudinal axis X of the residence time arrangement 10 can comprise one or more residence time levels 18. They in turn can each comprise at least one, in particular exactly one, residence time level channel 19 arranged transversely, in particular orthogonally, to the longitudinal axis X of the residence time arrangement 10, as can be seen in FIG. 5.

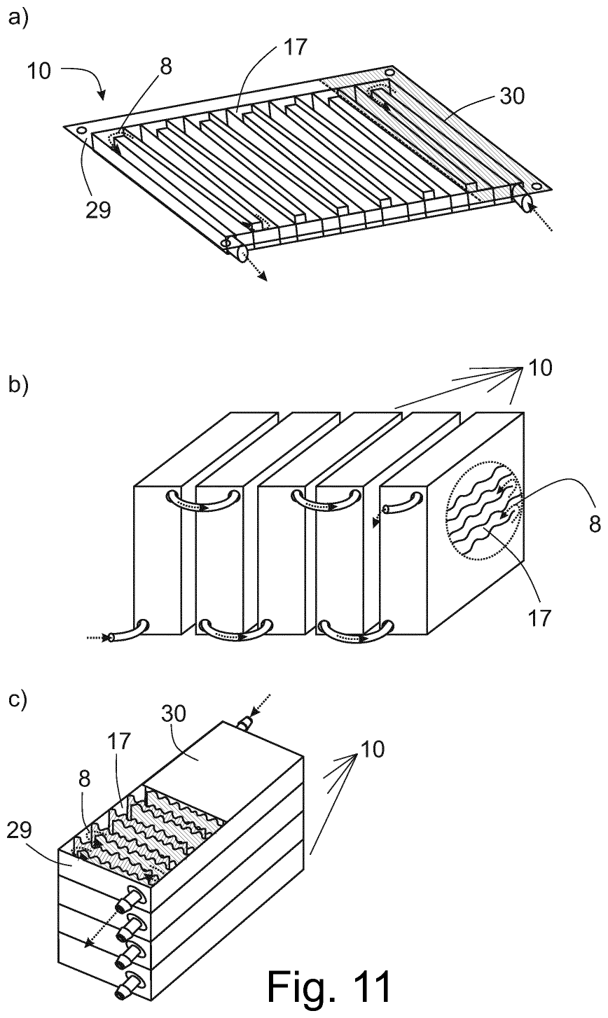

The respective residence time level 18 can be in the form of a preassembled or one-piece component. As can be seen in FIG. 5a) and b), said component can be substantially disk-shaped. Here, this means a design with a planar underside and a topside having ribs which separate the subsections of the respective residence time level channel 19 from one another. In some embodiments, a plurality of these one-piece components is fluidically and sealingly connectable to one other, in particular stackable on top of one other (FIG. 11c)), in some embodiments stackable on top of one another with an angular offset between adjacent one-piece components, in order to form the channel system 17, as depicted in FIG. 4. The angular offset between the adjacent one-piece components can be at least 45°, at least 90°, at least 1350, or 1800.

Here, the term “sealingly” means a seal with respect to the entry of air and the escape of the liquid stream 8. It is also possible that, in each of the residence time levels 18, single or multiple deflections of the third, reactive liquid stream 8 occur such as by at least 45°, by at least 90°, by at least 135°, or by 180°. Therefore, one residence time level channel 19 can comprise multiple parallel subsections. In some embodiments, the channel system 17 of the residence time arrangement 10 has an identical cross-sectional area over the predominant part of the channel, such as of all parallel subsections of the respective residence time level channel 19. Furthermore, the one-piece component can form an assembly unit (FIG. 5) which is arrangeable as a whole on or in the device 1. These components can be identical in design. A plurality of these one-piece components can be coupled via an interlocking, frictional and/or bonded connection. The design with multiple residence time levels 18 means that the incubation time of the first liquid stream 6 containing a target protein with the second, virus-inactivating liquid stream 7 can be varied and be adapted to the individual requirements.

When the device 1 is assembled as intended, the residence time arrangement 10 is sealingly connected via a interlocking, frictional and/or bonded connection to the head part 9, in particular to the inlet head part 15 and/or the outlet head part 16. Here, the head part 9, in particular the inlet head part 15 and/or the outlet head part 16, can be in the form of a cap or the like and be separably or nonseparably fitted onto the residence time arrangement 10. Here, “separably” means “nondestructively separable”, whereas “nonseparably” means “destructively” separable.

Here, the device 1 comprises, in some embodiments upstream of the residence time arrangement 10, at least one sensor 20 for measurement of a parameter of the third, reactive liquid stream 8. Additionally or alternatively, the device 1 can comprise, in some embodiments downstream of the residence time arrangement 10, at least one sensor 20 for measurement of a parameter of the fifth, resultant liquid stream 13. The at least one sensor 20 can measure at least one parameter from the group comprising, but not limited to, pH, conductivity, temperature, light absorption, light intensity, light scattering and/or other spectrophotometric properties. In the case of the design with a plurality of sensors 20, at least two thereof can measure the same parameter or different parameters.

Furthermore, the at least one sensor 20 can be integrated into the head part 9. n some embodiments, at least one sensor 20 is integrated into the inlet head part 15 and arranged downstream of the first mixer 4. Additionally or alternatively, at least one sensor 20 can be integrated into the outlet head part 16 and in particular arranged downstream of the second mixer 14. This can be achieved in each case either by a destructively separable integration of the at least one sensor 20 into the respective head part 9 or by a separable plug-in mechanism in which the sensor 20 can be inserted from the outside through a through opening in the respective head part 9 in order to fulfill the intended function.

In some embodiments, the device 1 comprises a separate inlet intermediate plate 21 between the inlet head part 15 and the residence time arrangement 10. Additionally or alternatively, the device 1 can comprise a separate outlet intermediate plate 22 between the residence time arrangement 10 and the outlet head part 16 for respective specific transfer of the third, reactive liquid stream 8.

Here, “separate” means components which have been produced independently of one another and which are put together during assembly to form a non-one-piece device 1.

Moreover, the inlet intermediate plate 21 and/or the outlet intermediate plate 22 can be in the form of components which are to be separately installed and which are also nondestructively or destructively separable from the device 1. Here, as depicted in FIGS. 2b), 4b), 6b) and 7b), the inlet intermediate plate 21 separates the interior of the inlet head part 15 from the interior of the residence time arrangement 10 and/or the outlet intermediate plate 22 separates the interior of the residence time arrangement 10 from the interior of the outlet head part 16. In some embodiments, the inlet intermediate plate 21 and/or the outlet intermediate plate 22 has a through opening 23 which, when the device 1 is assembled as intended, fluidically connects the interior of the respective head part 15, 16 to the interior of the residence time arrangement 10. The through opening 23 of the inlet intermediate plate 21 can be in particular coaxial in relation to the longitudinal axis X of the residence time arrangement 10, whereas the through opening 23 of the outlet intermediate plate 22 can be radially offset in relation to the longitudinal axis X of the residence time arrangement 10, in particular arranged at the radial edge of the residence time arrangement 10. For a particularly specific fluidic connection, the through opening 23 can be in the form of a single fluidic connection between the interior of the respective head part 15, 16 and the interior of the residence time arrangement 10, as depicted in FIGS. 2, 4, 6 and 7.

As can be seen in FIGS. 2a), 4a), 6a) and 7a), the components of the device 1 at least necessary for the intended function can form a functional group 24. Said functional group 24 can comprise in particular at least the inlet head part 15, the residence time arrangement 10 and the outlet head part 16. In some embodiments, the functional group 24 is in the form of a preassembled or one-piece unit. Therefore, the functional group 24 can form an assembly unit which is arrangeable as a whole on or in the device 1. Since said functional groups 24 can be identical in design, this allows a modular structure with a plurality of said functional groups 24. A plurality of said functional groups 24 can be coupled via an interlocking fluidic connection, frictional fluidic connection and/or bonded fluidic connection. The design with multiple functional groups 24 means that the incubation time of the first liquid stream 6 with the second, virus-inactivating liquid stream 7 can be varied and be adapted to the individual requirements.

It is also possible to bring the device 1, in some embodiments the residence time arrangement 10, to a temperature tailored to the respective product in order to quicken the chemical reaction, in particular to a temperature between 16° C. and 42° C. The flow rate can be controlled by, for example, a pump and thereby adapted to the individual process requirements, in particular a flow rate between 0.1 and 4 times the internal volume of the residence time arrangement 10 per hour. The material chosen for the proposed device 1 is a material compatible with a bioprocess, in particular with a biopharmaceutical process, such as metal, plastic, in particular polyamide (PA) plastics, polypropylene (PP), polyethylene (PET), acrylonitrile-butadiene-styrene copolymer (ABS) or polyvinylidene fluoride (PVDF), rubber and/or glass.

The flow direction in the coupling of multiple functional groups 24 can occur along the two directions of the longitudinal axis X of the residence time arrangement 10 that are possible in principle, in particular alternatingly from functional group 24 to functional group 24. Here, preference can be given to the flow direction from bottom to top, in particular against the gravitational direction, since it allows more effective venting of the device 1, thereby preventing air bubbles within the system and also consequently preventing impairments in the downstream process.

The virus-inactivating conditions can be chosen with the proposed device 1 such that virus inactivation can be realized by a factor of at least 1×10¹, at least 1×10³, or at least 1×10⁶, in particular irrespective of whether there is exactly one specific virus type, multiple virus types and/or a plurality of different virus types. The virus-inactivating conditions can be chosen such that less than 1 ppm, or less than 1 ppb, of the volume of the third, reactive liquid stream 8 has a residence time in the residence time arrangement 10 which is shorter than that which would be necessary for effective virus inactivation by a factor of at least 1×10¹, in particular of at least 1×10⁶. The proposed device 1 can be designed with or without feedback control.

Here, the term “feedback control” means the self-regulation of the activity of the system, in particular with regard to the size of the volume flow, on the basis of information about the effects of earlier activities, measured by means of data-connected sensors 20.

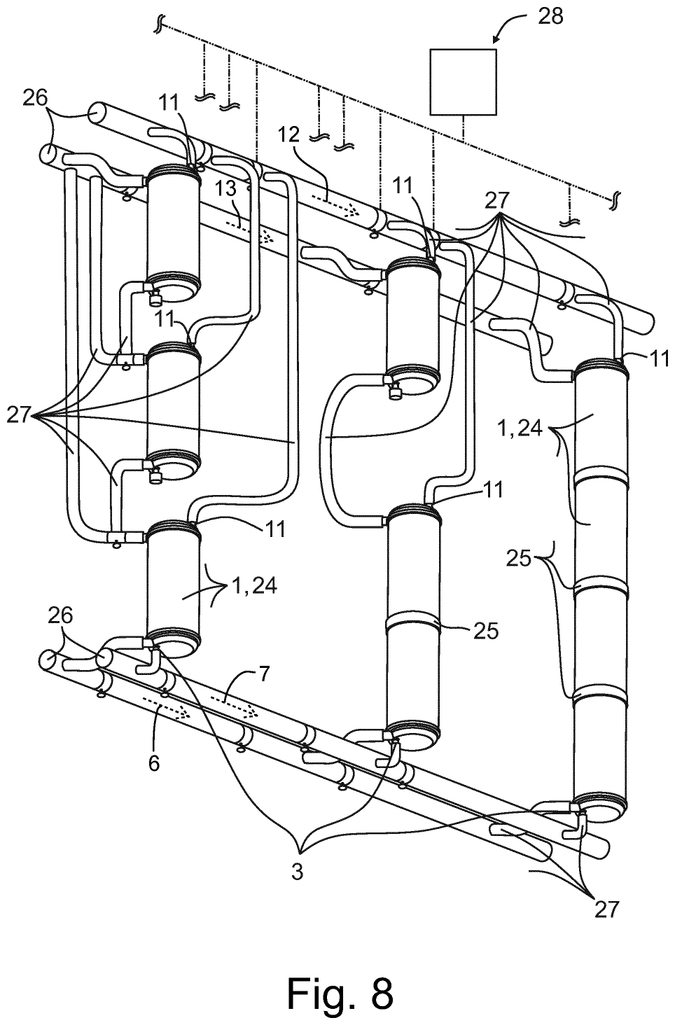

FIG. 8 shows that, for an individual adaptation of the virus inactivation process to different process requirements, the device 1 can comprise only a single functional group 24 or a plurality of parallelly and/or serially arranged functional groups 24 fluidically connected to one another. For example, a parallel arrangement means that it is possible, at the same flow rate, to increase the volume flow in the protein production process that is to undergo virus inactivation by means of the proposed device 1. A serial arrangement means that it is possible, at the same flow rate, to increase the residence time of the volume flow in the proposed device 1 that is to undergo virus inactivation or that it is possible, at the same residence time due to an increased flow rate, to likewise increase the volume flow that is to undergo virus inactivation. Multiple functional groups 24 of this kind can be fluidically and sealingly connected by means of a plug-in and/or screw mechanism or the like, which are realizable by a corresponding structural design of the functional group 24. Additional or alternatively, a connection element 25, in particular an adapter and/or flexible tube, which fluidically and sealingly connects at least two such functional groups 24 to one another can be provided, as can be seen in FIG. 8.

In the case of multiple parallel functional groups 24, an acid, for example, can be supplied through a particular central line 26 of a plurality of central lines 26 and/or through particular individual lines 27 by means of, in each case, the second fluid inlet 3 of the respective functional group 24.

Here, as depicted in FIG. 8, a plurality of central lines 26, each of which transports a different liquid stream (6, 7, 12, 13), is provided. Merely as an example, what is depicted here is that the bottom-left central line 26 transports the first liquid stream 6 containing a target protein. The bottom-right central line 26 transports the second, virus-inactivating liquid stream 7. The top-right central line 26 transports the fourth, neutralizing liquid stream 12, whereas the top-left central line 26 transports the fifth, resultant liquid stream 13.

Similarly, in the case of multiple parallel functional groups 24, a base, for example, can be supplied through a particular central line 26 of a plurality of central lines 26 and/or through particular individual lines 27 by means of, in each case, the third fluid inlet 11 of the respective functional group 24.

In the case of multiple serial functional groups 24, an acid can be supplied either through a central line 26 only into the fluidically first functional group of the serial functional groups 24 or through individual lines 26 into each of the serial functional groups 24 by means of, in each case, the second fluid inlet 3. Furthermore, in the case of multiple serial functional groups 24, abase can be supplied either through a central line 26 only into the fluidically last functional group of the serial functional groups 24 or through individual lines 27 into each of the serial functional groups 24 by means of, in each case, the third fluid inlet 11. It should be noted that, for example, detergents and other substances suitable for virus inactivation can also be used instead of acid or base.

Furthermore, a control arrangement 28 of the device 1 as depicted in FIG. 8 can be used to connect or disconnect, from the plurality of functional groups 24, individual functional groups 24 or a group of functional group 24, in particular each functional group 24. A group of functional groups 24 can be, for example, a string of serially and/or parallelly arranged functional groups 24 fluidically connected to one another.

Here, “connected” or “connectable” and “disconnected” or “disconnectable” means that it is possible to individually pass through each of these functional groups 24 as needed depending on the process requirements and the desired volume flow and/or desired residence time in the device 1, thereby allowing particular flexibility in process planning. Here, connection or disconnection is achieved by valve control and/or pump control. Here, said connection or disconnection can be controlled by at least one component of the control arrangement 28 intended for this purpose, for example can be achieved by a computer, a server, a cloud application, a mobile app, a tablet or smartphone or a combination. Furthermore, connection or disconnection can be achieved from outside the room or the building or the like in which the other components of the device 1 necessary for the intended function are located. In addition, a data connection with at least one sensor 20 can be establishable and/or a feedback control can be implementable by means of control arrangement 28.

Here, the residence time arrangement 10, the head part 9, in particular the inlet head part 15 and/or the outlet head part 16, the inlet intermediate plate 21 and/or the outlet intermediate plate 22 are produced, in particular individually or together, in a plastics injection-molding process, in a 3D printing process or by machining technologies, in particular milling.

In terms of implementing a single-use concept as discussed above, preference can be given to the device 1 as a whole or at least the residence time arrangement 10, the inlet head part 15, the outlet head part 16, the inlet intermediate plate 21 and/or the outlet intermediate plate 22 being designed as a single-use component or as single-use components. In various embodiments, at least one of these components is made of a plastics material at least in part, such as predominantly.

In a further teaching that is of independent significance, there is provided a residence time arrangement 10 for provision of a predefined minimum residence time of a reactive liquid stream 8 during continuous virus inactivation in a device 1 for continuous virus inactivation, in particular in a proposed device 1, in particular during an antibody production process. When the residence time arrangement 10 is assembled as intended, a liquid stream 8 containing a target protein and having predefined, virus-inactivating conditions is introducible into the residence time arrangement 10. Reference may be made in this respect to all discussions relating to the proposed device 1.

What is essential in the case of the proposed residence time arrangement 10 is then, first of all, that the residence time arrangement 10 comprises an internal channel system 17 for provision of a minimum residence time of the reactive liquid stream 8 within the residence time arrangement 10. The internal channel system 17 comprises at least one channel through which the reactive liquid stream 8 flows during the continuous virus inactivation. Furthermore, it is essential that the liquid stream 8 is deflected at least once within the respective channel.

Here, the term “deflected” means that the course of the liquid stream changes, for example from a straight course into a curved course or from a curved course into a more curved course. Following the deflection, the liquid stream can return to a straight course or a less curved course. A course of the liquid stream on a path having a constant radius of curvature, for example a spiral path, is therefore not a deflection in the present sense.

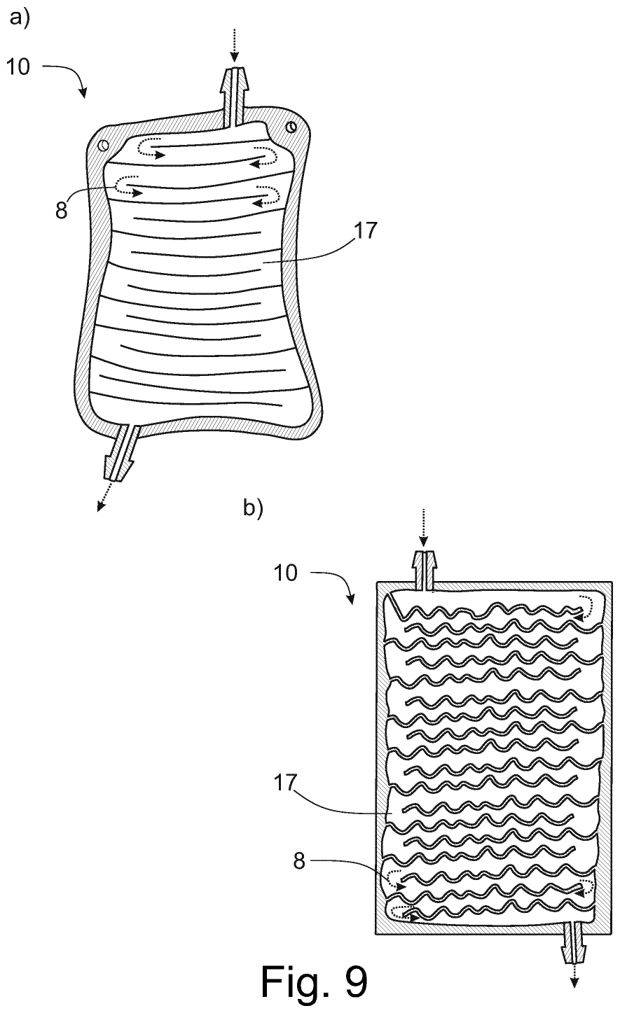

Here, as shown in FIGS. 9, 10 and 11, the liquid stream 8 is deflected within the respective channel in such a way that its subsequent flow direction runs transversely to the previous flow direction. In some embodiments, the liquid stream 8 is deflected within the respective channel by at least 45°, by at least 90°, by at least 135°, or by 180°.

An exemplary embodiment according to FIG. 9 relates to a residence time arrangement 10 in which the liquid stream 8 is deflected within the respective channel at least once in such a way that its subsequent flow direction runs transversely to the previous flow direction. The residence time arrangement 10 as such can be in the form of a bag (FIG. 9a)), such as a disposable bag. It can be made of biocompatible disposable materials. For bioprocess applications, preference can be given to using three-ply composite films as disposable material. They can consist of a mechanical support layer (e.g., PET, LDPE), a gas-impermeable barrier layer (e.g., EVA, PVDC) and a contact layer (e.g., EVA, PP). In some embodiments, the residence time arrangement 10 in the form of a bag, in particular a disposable bag, is produced by heat-sealing of the plies of a composite film. For many applications, the disposable materials can be certified by a drug authorization authority. Alternatively, the residence time arrangement 10 can be in the form of a cassette (FIG. 9b) and/or a reusable component, made of in particular glass or stainless steel.

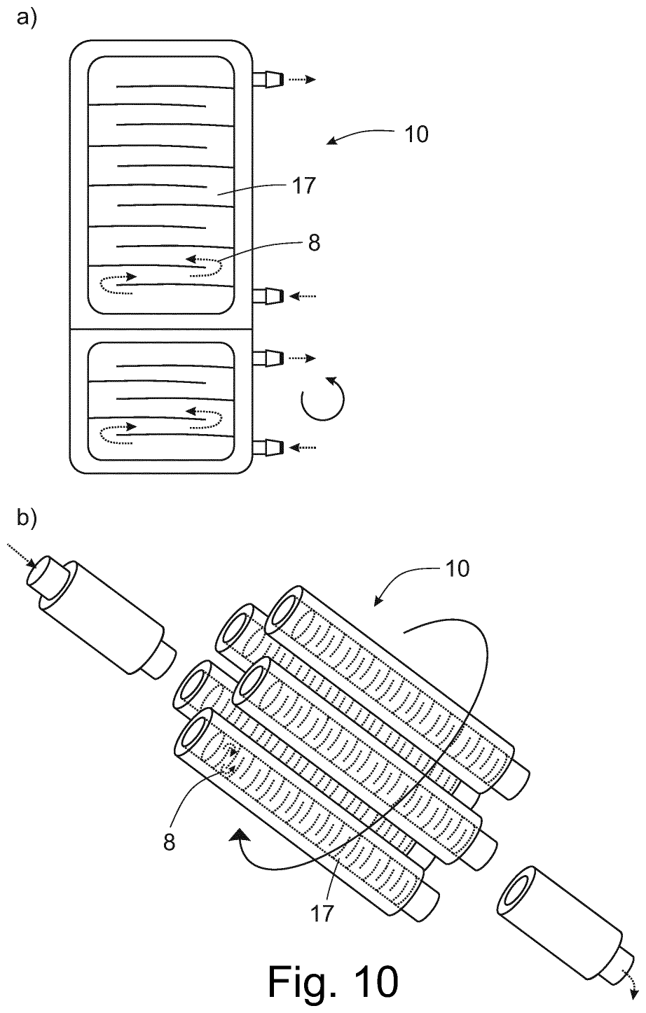

As depicted in FIG. 10a), the residence time arrangement 10 can comprise at least two chambers, wherein one chamber, here the first chamber, and whichever other chamber is adjacent, here the second chamber, are fluidically connectable to one another. The first chamber can serve for mixing, in which the reactive liquid stream 8 can be recirculated until, for example, the desired target value, in particular pH target value, has been set. The first chamber is then fluidically connected to the second chamber, so that the reactive liquid stream 8 resides in the second chamber until the intended minimum residence time for virus inactivation has been reached.

Here, multiple residence time arrangements 10 can be in the form of a “revolver system” (FIG. 10b)). A plurality of residence time arrangements 10 is filled sequentially with the reactive liquid stream 8, after the at least one parameter of the reactive liquid stream 8, in particular the pH of the reactive liquid stream 8, has been initially set in a mixing module upstream of the residence time arrangement 10. After a first residence time arrangement 10 has been filled, the revolver can rotate in order to bring into position a further residence time arrangement 10 for filling. It is then filled while the virus inactivation is already taking place in the first residence time arrangement 10. After the virus inactivation has been carried out, the contents of the residence time arrangement 10 can be transferred to a downstream neutralization module in which the virus-inactivating conditions are terminated, such as in which the pH is neutralized. This design form allows sequential filling of a multiplicity of residence time arrangements 10 and ensures increased efficiency, since parameter setting, filling, virus inactivation and neutralization can take place in parallel, at least in part.

A further exemplary embodiment according to FIG. 11a) likewise relates to a residence time arrangement 10 in which the liquid stream 8 is deflected within the respective channel at least once in such a way that its subsequent flow direction runs transversely to the previous flow direction. Here, the residence time arrangement 10 is of a two-part design 29, 30 (FIGS. 11a) and 11c)). The first residence time arrangement part 29 forms the internal channel system 17, which can be produced by deep drawing.

According to DIN 8584, “deep drawing” is the tensile compressive forming of a blank into a hollow body open on one side. In the case of production by deep drawing, the residence time arrangement 10 can be made of metal, such as stainless steel or sheet metal.

The second residence time arrangement part 30 can be in the form of a flat plate or film and forms the residence time arrangement 10 by a sealing connection with the first residence time arrangement part 29. As already explained above, the residence time arrangement 10 can be made of biocompatible disposable materials. To produce a one-piece residence time arrangement 10, the first and the second residence time arrangement part 29, 30 can be composed of film layers can be heat-sealed to one another. At least one fluid inlet and at least one fluid outlet can in principle be arranged on the same, adjacent or opposite sides of the residence time arrangement 10. Further production options for proposed residence time arrangements 10 can be plastics injection molding, 3D printing or machining technologies, in particular milling.

Here, multiple residence time arrangements 10 can be stacked, as depicted in FIG. 11c). Additionally or alternatively, they can be operated in parallel (FIGS. 8 and 11c)) or in series (FIGS. 8 and 11b)).

The proposed residence time arrangement 10 can be further designed in different ways, such as by one or more of the features which have been described above in relation to the proposed device 1. Irrespective of the design of the device 1, each of these features and each combination of features from these features is respectively suitable for advantageously developing the proposed residence time arrangement.

In a further teaching that is of independent significance, there is provided a residence time arrangement 10 for provision of a predefined minimum residence time of a reactive liquid stream 8 during continuous virus inactivation in a device 1 for continuous virus inactivation, in particular in a proposed device 1, in particular during an antibody production process. When the residence time arrangement 10 is assembled as intended, a liquid stream 8 containing a target protein and having predefined, virus-inactivating conditions is introducible into the residence time arrangement 10. Reference may be made in this respect to all discussions relating to the proposed device 1.

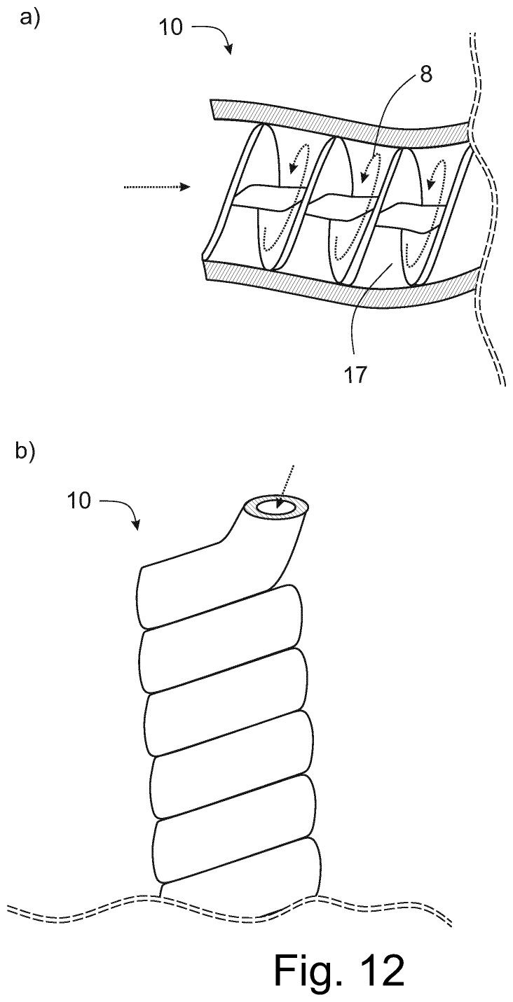

What is essential in the case of the proposed residence time arrangement 10 is then that the residence time arrangement 10 comprises an internal channel system 17 for provision of a minimum residence time of the reactive liquid stream 8 within the residence time arrangement 10. Here, according to FIG. 12, the internal channel system 17 comprises at least one channel through which the reactive liquid stream 8 flows during the continuous virus inactivation. Furthermore, it is essential that the at least one channel is helically designed for provision of a predefined minimum residence time (FIG. 12a)), and that the residence time arrangement 10 is helically arranged in the intended state (FIG. 12b)).

Preference can be given to the residence time arrangement 10 being made of a rigid or flexible material, and further preference can be given to the residence time arrangement 10 being in the form of a tube or flexible tube. The material used can be biocompatible plastics, such as so that the proposed residence time arrangement 10 is in the form of a disposable component. Alternatively, the residence time arrangement can be in the form of a reusable component, such as made of metal, such as stainless steel, or glass.

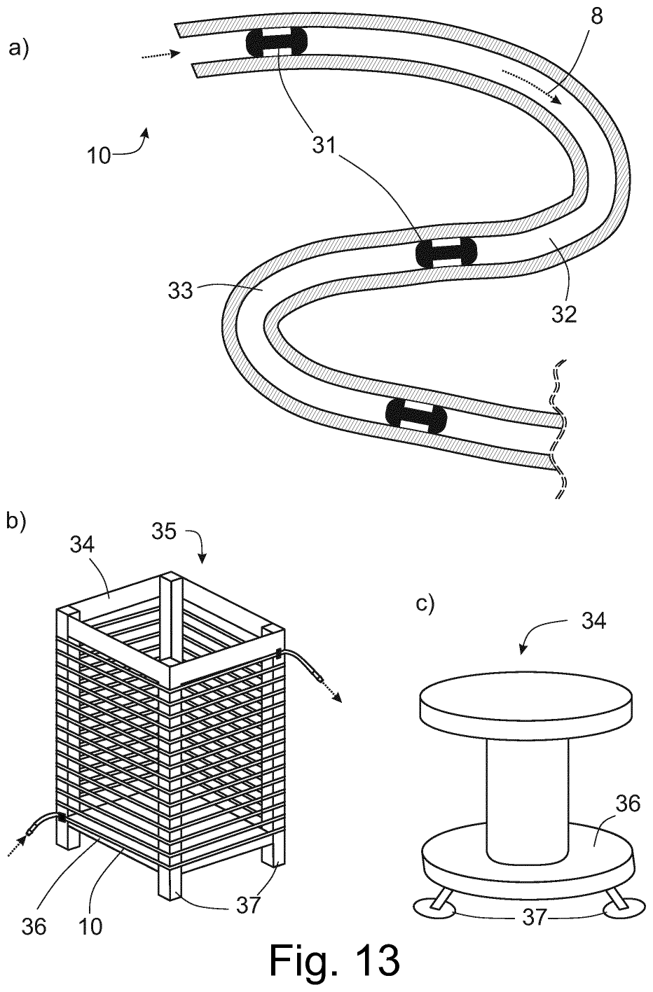

Here, as shown in FIG. 13a), the residence time arrangement 10 has at least one pig 31 in the internal channel system 17, such as in the at least one channel of the internal channel system 17.

A “pig” is a cleaning and/or inspection device for use in lines, here for cleaning and/or inspection of the residence time arrangement 10. Furthermore, a pig 31 allows clean separation between successive product batches or, depending on the consistency of the product, support of transport itself.

The pig 31 fills the cross-section of the line and either travels through the line with the product stream or must be pushed through the line by application of pressure. Pigging technology provides not the pig 31, but also pig traps, which are incorporated in the internal channel system 17 and through which the pig 31 can be inserted into the lines and placed under pressure from behind, but can also be removed after completion of the intended test section.

In some embodiments, volume sections 32, 33 which differ in at least one parameter, in particular with regard to pH, can be cleanly separated from one other by the use of at least one pig 31 within the residence time arrangement 10. This allows the provision of a minimum residence time of the reactive liquid stream 8 within the residence time arrangement 10, wherein separate volume sections 32, 33 are subsequently combinable again by it being possible to remove the separating pigs 31 by means of pig traps. This can allow, firstly, the separation and, secondly, the combination of at least two volume sections 32, 33, such as at least two liquid streams 6, 7, 8, 12, 13. As a result, a first volume component 32, in particular a liquid stream 6 containing a target protein, can initially be separate from a second volume component 33, in particular from a virus-inactivating liquid stream 7, and then be combined in order to generate a third, reactive liquid stream 8. Furthermore, said third, reactive liquid stream 8, after a minimum residence time has passed within the residence time arrangement 10, can be combined with a further volume component, in particular with a neutralizing liquid stream 12, for neutralization of the virus-inactivating conditions to form a fifth, resultant liquid stream 13.

Here, as shown in FIGS. 13b) and 13c), the at least one residence time arrangement 10 is assigned a corresponding holding arrangement 34. When assembled as intended, the residence time arrangement 10 is arranged helically on the holding arrangement 34, so that the residence time arrangement 10 and the holding arrangement 34 assigned thereto jointly form a residence time system 35 (FIG. 13 b).

Here, the term “holding arrangement” means any structure which allows shaping of the residence time arrangement 10, such as in such a way that the residence time arrangement 10 is arranged on the holding arrangement 35.

Here, the holding arrangement 35 has a rectangular, such as square, base surface 36, as depicted in FIG. 13b). Alternatively, the holding arrangement 35 has a rounded, such as round or oval, base surface 36, as depicted in FIG. 13c). The respective holding arrangement 35 can comprise support elements 37, in particular casters or support feet, which provide for increased mobility and clearance from the ground, so that in particular any sensors, cables and/or lines of any kind are not damaged.

The proposed residence time arrangement 10 can be further designed in different ways, such as by one or more of the features which have been described above in relation to the proposed device 1. Irrespective of the design of the device 1, each of these features and each combination of features from these features is respectively suitable for advantageously developing the proposed residence time arrangement.

In a further teaching that is of independent significance, there is provided a method for continuous virus inactivation during a protein production process, in particular an antibody production process, using a proposed device 1 and optionally a proposed residence time arrangement 10. In some embodiments, a first liquid stream 6 containing a target protein is introduced into the device 1 through the first fluid inlet 2 and combined in a precisely predefined volume ratio with a second, virus-inactivating liquid stream 7 introduced into the device 1 through the second fluid inlet 3 to form a third, reactive liquid stream 8. It is conducted through the first mixer 4 for mixing in order to generate predefined, virus-inactivating conditions, wherein the third, reactive liquid stream 8 is generated in the inlet head part 15 and wherein a minimum residence time of the third, reactive liquid stream 8 within the device 1 is provided by means of the residence time arrangement 10. Reference may be made in this respect to all discussions relating to the proposed device 1.

Here, the virus-inactivating conditions in the third, reactive liquid stream 8 can be neutralized in the proposed method. This can be achieved in the outlet head part 16 by means of a fourth, neutralizing liquid stream 12 introduced into the device 1.

Furthermore, the proposed method can be carried out in combination with chromatography methods, in particular affinity chromatography and ion-exchange chromatography methods, such as with continuous chromatography methods. Furthermore, the proposed method can be performed in combination with filtration methods, in particular with tangential flow filtration methods. In principle, the proposed device 1 and the proposed method can be used in combination with all purification, filtration, chromatography, separation, centrifugation, concentration and/or sedimentation methods or other methods which can be classified under upstream or downstream process of protein products.

In process engineering, “upstream process” refers to all steps in relation to cell line, seed train development, media development, optimization of growth kinetics and the cell cultivation or fermentation process itself, and also the corresponding in-process controls.

In process engineering, “downstream process” refers to all methods used to remove and purify fermentation products from a fermentation broth of a biotechnological process. This term encompasses mechanical, thermal, electrical and physicochemical methods.

In a further teaching that is of independent significance, the use of a proposed device 1 for implementation of a protein production process, in particular an antibody production process, is provided for. Reference may be made in this respect to all discussions relating to the proposed device 1.

A variant of the use of the proposed device 1 consists in the inlet head part 15, the residence time arrangement 10 and/or the outlet head part 16 being used as a single-use component.

1. A device for continuous virus inactivation during a protein production process, comprising:
a first and a second fluid inlet, each configured to introduce a liquid stream into the device,
a first mixer, configured to mix a liquid stream, and
a fluid outlet, configured to discharge a liquid stream from the device, wherein, when the device is assembled as intended, a first liquid stream containing a target protein is introducible into the device through the first fluid inlet and is combinable in a precisely predefined volume ratio with a second, virus-inactivating liquid stream introducible into the device through the second fluid inlet to form a third, reactive liquid stream which is conducted through the first mixer for mixing in order to generate predefined, virus-inactivating conditions,
wherein the device comprises a head part for combination of two liquid streams, one of which is the liquid stream containing the target protein, wherein the device comprises, downstream of the first mixer and upstream of the fluid outlet, a residence time arrangement fluidically connected to the head part for provision of a minimum residence time of the third, reactive liquid stream within the device, wherein the head part and the residence time arrangement are rigidly fastened to each other, and wherein the head part is arranged on an end face of the residence time arrangement.

2. The device as claimed in claim 1, wherein the device comprises, downstream of the first mixer and the residence time arrangement, a third fluid inlet, configured to introduce a liquid stream into the device, and in that, when the device is assembled as intended, the third, reactive liquid stream is combinable with a fourth, neutralizing liquid stream introducible through the third fluid inlet to form a fifth, resultant liquid stream in order to neutralize the virus-inactivating conditions.

3. The device as claimed in claim 1, wherein the device comprises, downstream of the third fluid inlet, a second mixer, configured to mix the fifth, resultant liquid stream.

4. The device as claimed in claim 1, wherein the head part is an inlet head part for generation of the third, reactive liquid stream and in that the inlet head part is arranged at one end of the residence time arrangement in order to generate the virus-inactivating conditions.

5. The device as claimed in claim 1, wherein the head part is an outlet head part for generation of the fifth, resultant liquid stream and wherein the outlet head part is arranged at the other end of the residence time arrangement in order to neutralize the virus-inactivating conditions.

6. The device as claimed in claim 1, wherein the first and/or the second fluid inlet and/or the first mixer is integrated into the inlet head part, and/or wherein the third fluid inlet and/or the second mixer is integrated into the outlet head part.

7. The device as claimed in claim 1, wherein the first liquid stream containing a target protein and the second, virus-inactivating liquid stream are combined upstream of the first mixer or in the first mixer, and/or wherein the third, reactive liquid stream and the fourth, neutralizing liquid stream are combined upstream of the second mixer or in the second mixer.

8. The device as claimed in claim 1, wherein the residence time arrangement is in the form of a substantially cylindrical or cuboid body and/or in the form of a one-piece body.

9. The device as claimed in claim 1, wherein the residence time arrangement comprises an internal channel system which is arranged at least largely parallel to the longitudinal axis of the residence time arrangement and/or an internal channel system which is arranged at least largely transversely to the longitudinal axis of the residence time arrangement and/or an internal channel system which is at least largely arranged as a worm shaft and/or an internal channel system which is a least largely arranged as a cascade.

10. The device as claimed in claim 9, wherein the internal channel system which is arranged at least largely transversely to the longitudinal axis of the residence time arrangement comprises one or more residence time levels, each of which comprises at least one residence time level channel arranged transversely to the longitudinal axis of the residence time arrangement.

11. The device as claimed in claim 9, wherein the respective residence time level is in the form of a preassembled or one-piece component.

12. The device as claimed in claim 1, wherein the residence time arrangement is sealingly connected via an interlocking, frictional and/or bonded connection to the head part.

13. The device as claimed in claim 1, wherein the device comprises at least one sensor for measurement of a parameter of the third, reactive liquid stream and/or at least one sensor for measurement of a parameter of the fifth, resultant liquid stream.

14. The device as claimed in claim 13, wherein the at least one sensor is integrated into the head part and the at least one sensor is arranged downstream of the first mixer and/or wherein the at least one sensor is integrated into the outlet head part the at least one sensor is arranged downstream of the second mixer.

15. The device as claimed in claim 1, wherein the device comprises a separate inlet intermediate plate between the inlet head part and the residence time arrangement and/or a separate outlet intermediate plate between the residence time arrangement and the outlet head part for the respective specific transfer of the third, reactive liquid stream.

16. The device as claimed in claim 1, wherein the components of the device at least necessary for the intended function, form a functional group wherein the functional group is in the form of a preassembled or one-piece unit.

17. The device as claimed in claim 16, wherein the device comprises only a single functional group or a plurality of parallelly and/or serially arranged functional groups fluidically connected to one another.

18. The device as claimed in claim 1, wherein the residence time arrangement, the head part, the inlet intermediate plate and/or the outlet intermediate plate are produced in a plastics injection-molding process or in a 3D printing process.

19. A residence time arrangement for provision of a predefined minimum residence time of a reactive liquid stream during continuous virus inactivation in a device for continuous virus inactivation, wherein, when the residence time arrangement is assembled as intended, a liquid stream containing a target protein and having predefined, virus-inactivating conditions is introducible into the residence time arrangement,
wherein the residence time arrangement comprises an internal channel system for provision of a minimum residence time of the reactive liquid stream within the residence time arrangement, in that the internal channel system comprises at least one channel through which the reactive liquid stream flows during the continuous virus inactivation, and wherein the liquid stream is deflected at least once within the respective channel.

20. The residence time arrangement as claimed in claim 19, wherein the liquid stream is deflected within the respective channel in such a way that its subsequent flow direction runs transversely to the previous flow direction.

21. The residence time arrangement as claimed in claim 19,
wherein the device comprises, downstream of the first mixer and the residence time arrangement, a third fluid inlet, configured to introduce a liquid stream into the device, and in that, when the device is assembled as intended, the third, reactive liquid stream is combinable with a fourth, neutralizing liquid stream introducible through the third fluid inlet to form a fifth, resultant liquid stream in order to neutralize the virus-inactivating conditions.

22. A residence time arrangement for provision of a predefined minimum residence time of a reactive liquid stream during continuous virus inactivation in a device for continuous virus inactivation, wherein, when the residence time arrangement is assembled as intended, a liquid stream containing a target protein and having predefined, virus-inactivating conditions is introducible into the residence time arrangement,
wherein the residence time arrangement comprises an internal channel system for provision of a minimum residence time of the third, reactive liquid stream within the residence time arrangement, in that the internal channel system comprises at least one channel through which the reactive liquid stream flows during the continuous virus inactivation, in that the at least one channel is helically designed for provision of a predefined minimum residence time, and in that the residence time arrangement is helically arranged in the intended state.

23. The residence time arrangement as claimed in claim 22, wherein the residence time arrangement is made of a rigid or flexible material.

24. The residence time arrangement as claimed in claim 22, wherein the residence time arrangement has at least one pig in the internal channel system.

25. The residence time arrangement as claimed in claim 22, wherein the at least one residence time arrangement is assigned a corresponding holding arrangement, wherein the residence time arrangement is arranged helically on the holding arrangement when assembled as intended, so that the residence time arrangement and the holding arrangement assigned thereto jointly form a residence time system.

26. The residence time arrangement as claimed in claim 25, wherein the holding arrangement has a rectangular or rounded base surface.

27. The residence time arrangement as claimed in claim 22, wherein the device comprises, downstream of the first mixer and the residence time arrangement, a third fluid inlet, configured to introduce a liquid stream into the device, and in that, when the device is assembled as intended, the third, reactive liquid stream is combinable with a fourth, neutralizing liquid stream introducible through the third fluid inlet to form a fifth, resultant liquid stream in order to neutralize the virus-inactivating conditions.

28. A method for continuous virus inactivation during a protein production process, in particular an antibody production process, using a device as claimed in claim 1, wherein a first liquid stream containing a target protein is introduced into the device through the first fluid inlet and is combined in a precisely predefined volume ratio with a second, virus-inactivating liquid stream introduced into the device through the second fluid inlet to form a third, reactive liquid stream which is conducted through the first mixer for mixing in order to generate predefined, virus-inactivating conditions, wherein the third, reactive liquid stream is generated in the inlet head part and wherein a minimum residence time of the third, reactive liquid stream within the device is provided by the residence time arrangement.

29. The method as claimed in claim 28, wherein the virus-inactivating conditions in the third, reactive liquid stream are neutralized in the outlet head part a fourth, neutralizing liquid stream introduced into the device.

30. The method as claimed in claim 28, wherein it is performed in combination with chromatography methods and/or in combination with filtration methods.

31. The use of a device as claimed claim 1 for implementation of a protein production process.
